(19) United States
(12) Patent Application Publication
(10) Pub. No.: US 2026/0341509 A1
(43) Pub. Date: Oct. 29, 2026

(54) Information System to Prevent Diseases

(71) Applicant: Robert Norton, Temple, TX (US)

(72) Inventor: Robert Norton, Temple, TX (US)

(21) Appl. No.: 19/192,343

(22) Filed: Apr. 29, 2025

(51) Int. Cl. G16H 50/20; G16H 10/60; G16H 50/30; G16H 50/70; G16H 70/40

(52) U.S. Cl. CPC G16H 50/20; G16H 10/60; G16H 50/30; G16H 50/70; G16H 70/40

(57) ABSTRACT

A system and method for identifying potential root causes of diseases and other health conditions by correlating large-scale consumer product purchase data, ingredient data, and medical records. The invention aggregates Electronic Medical Records (EMRs), purchase histories, and ingredient databases, applying statistical analysis, Machine Learning (ML), and Artificial Intelligence (AI) to detect significant correlations between products, ingredients, and medical diagnoses and treatments. By generating dangerousness indices, it enables proactive disease prevention, regulatory support, and consumer safety alerts. The system dynamically updates with new data, offering real-time public health benefits globally.

Information technology system and processes for identifying root causes of diseases that are caused by food, ingredients, products, and other environmental chemicals and factors by comparing vast amounts of purchases with medical records and chemical ingredients databases. This straddles information technology and medical research data.

### FIELD OF THE INVENTION

The present invention relates to health information, information technology systems, purchasing data (i.e. grocery stores), and AI to detect dangerous foods, ingredients and other correlations. This invention requires extensive knowledge of several fields including medical research, Electronic Medical Records (EMR) systems, computer science, and packaged goods and grocery industries information systems. By aggregating massive amounts of data from these three main sources, we can find root causes of diseases caused by packaged goods products, ingredients, and combinations thereof that are hidden by long separation in exposure and disease detection.

For example, some cancers can have a ten- or twenty-year latency after exposure to certain chemicals. Our system allows aggregation of these various databases: 1. Consumer purchase data (mainly from grocery stores)
2. Medical records with diagnoses of illness
3. Food ingredients in packaged goods from ingredient labels

This system can therefore potentially be used to:
Save tens of millions of lives via disease prevention
Prevent trillions in medical treatment costs and the associated pain and suffering
Multiply the effectiveness of billions in research dollars by focusing research on highly likely causes, instead of theories based on molecular biology alone

Research shows that much cancer is caused by chemicals in our food and there is no good process to detect and eliminate these sources in advance of putting new chemicals into the food chain.

### Technical Field to Which the Invention Pertains

The invention is a large data processing system, often called “Big Data,” designed to merge data from many specific sources. The system is enhanced by statistical knowledge, brute force comparison to seek correlations, and/or AI tools like Deep Learning (DL) and Machine Learning (ML) and can deal with massive datasets.

The goal is to use such massive amounts of information that the worst offenders for products consumed can be easily identified for purposes of further research to prove causality. This will require millions of person-years of data on both products purchased and medical history.

Specific legislation and privacy law modifications may be needed. This will provide a feedback loop to eliminate dangerous products and chemicals from our food supply and environment.

The system can also identify products that enhance health by showing, for example, that people who eat avocados live longer on average.

### BACKGROUND OF THE INVENTION

There is no relevant background other than the idea that came to the inventor after decades of cross-industry consulting experience in fields such as medicine, retail, brain scanning, packaged goods, and wellness sectors.

The approach becomes more practical over time due to:
Declining computing costs
Expansion of “Big Data”
Need for legislative action to handle privacy
Large funding requirements to develop massive databases

The invention seeks to integrate massive datasets to identify the hidden root causes of diseases across industries.

### Why is This Invention Non-obvious and Novel

### This Invention Spans:

Medicine
IT
Grocery and consumer goods industries

Deep experience in all these fields is required to even conceive of this system. The delay between exposure to chemicals and disease onset (often 5, 10, or 20 years) hides causality and makes traditional research methods ineffective. The inventor's unique background as a software engineer, development architect, Chief Technical Officer, serial entrepreneur, and consultant across 45 industries-enabled the creation of this system and method. Traditional researchers and developers lack the full cross-disciplinary perspective necessary to invent this system.

### PRIOR ART

A thorough search of the U.S. Patent and Trademark Office (USPTO) and global Google patent systems revealed no prior art comparable to this invention.

### Historical Examples Include:

Agent Orange exposure studies
Love Canal environmental disaster investigations
Erin Brockovich's litigation
“A Civil Action” litigation

### These Efforts:

Focused on known toxins and specific affected populations.
Were manual, slow, and after-the-fact.
Required human detection of illness clusters first.

### This Invention is:

This paragraph is intentionally left blank.”
Top-down: Finds correlations before mass illness.
Automated: Billions of combinations checked by algorithms.
Massively broader: All products, all ingredients, all diseases.
Impossibility of Traditional Research:
250,000+ grocery products
75,000+ plant ingredients
3,000+ artificial chemicals
750 million potential food combinations (minimum)
562,500 trillion permutations if combining just two ingredients!

Thus, manual testing of every possible harmful combination is impossible without this invention.

### Research on Statistical Databases and Tools to Cure Diseases

### Major Statistical Tools Used in Biomedical Research Include:

R: Open-source clinical trials/statistics
SAS: Enterprise-grade statistical platform
Stata: Biostatistics software
SPSS: Social and medical sciences analysis
GraphPad Prism: Biomedical publishing tool
MATLAB: Medical imaging signal processing
JMP (SAS): Pharma quality control
REGSTATTOOLS (NIH): Cancer burden estimation
   All of these Tools:
Are focused on individual studies.
Require human researchers to formulate hypotheses.
Do not aggregate product purchases+medical records for automated detection.

### Major Health & Biomedical Databases:

### Key Medical Databases Include:

RDCA-DAP (FDA Rare Disease Cure Accelerator)
AHRQ Data Tools
HealthData.gov
Human Mortality Database
MIMIC-IV (ICU database)
GenBank (NIH gene sequencing)
ClinicalTrials.gov
UK Biobank
   Again, these:
Focus on disease progression, treatment, genetics.
Do not correlate consumer product consumption with disease emergence.
   Platforms with AI/ML+Statistical Focus:

AI/ML research platforms:
KNIME: Biomarker discovery
Orange: Visual data mining
H2O.ai: Predictive analytics
Google BigQuery+Looker: Syndromic surveillance
Paragraph intentionally left blank

While powerful, these platforms:
Assist researchers to study what they already suspect.
Do not independently discover hidden correlations between products and medical outcomes.

### Emerging and Specialized Software & Tool Platforms

OHDSI/OMOP: Data model standardization
BioPortal: Biomedical ontology tools
TransSMART: Knowledge management platform

These focus on:
Supporting traditional, hypothesis-driven research.
Not identifying root causes of disease statistically or historically.

This line intentionally left blank

### Software and Database Tools to Facilitate Medical Research

Large EMR platforms include:
Epic Systems
Cerner
Praxis EMR
   Government databases:
All of Us Research Program
OPCRD (UK)
HCUP (AHRQ)
OpenSAFELY (UK)
PCORnet
Truveta

### APIs:

FHIR (HL7 Standard)
Consumer Health Data:
Apple HealthKit
Google Fit
Again, none of these:
Merge product purchase data with medical records.
Seek root cause prevention on a massive scale.

### CONCLUSION

This invention is novel, non-obvious, and essentially unprecedented. Unlike prior tools and databases that:
Support isolated, narrow studies
Rely on human-driven hypothesis creation
Are limited by funding, scope, and time

This invention:
Automatically finds hidden correlations between products, ingredients, and diseases.
Can perform trillions of comparisons in reasonable time using modern computing power.
Will dramatically accelerate health discoveries.
Will prevent millions of unnecessary deaths and suffering.
Will save trillions in healthcare costs globally over decades.
   Problems with Prior Art

There are no direct prior art solutions that:
Integrate consumer product consumption data with EMRs.
Seek root cause prevention across all diseases.
Use AI and brute-force computation for top-down pattern discovery at massive scale.

Prior research was limited to:
Known exposures
Manual studies
Small group analysis after disease outbreaks

Manual studies are retrospective and narrow. This invention is prospective, broad, and continuous.

### SUMMARY OF THE INVENTION

This invention uses vast databases to correlate:
1. Consumer product consumption
2. Ingredient exposure
3. Medical outcomes (positive or negative)
4. Data sources include:
5. Electronic Medical Records (diagnosis, treatment, outcome)
6. Consumer purchase histories
7. Detailed product ingredient databases

By aggregating and correlating these sources, the invention:
Detects hidden causes of diseases.
Identifies harmful products before widespread harm.
Identifies beneficial products that improve health.

Massive computational ability and AI can now test billions or trillions of product-ingredient-disease combinations affordably and quickly.

### General Description

The invention aggregates and cleans:
EMRs across many healthcare providers
Product purchase records across retailers
Ingredient databases across manufacturers

Key process steps:
Normalization of disparate data formats
De-duplication of medical records
Mapping product purchase histories to individual medical outcomes
Statistical and AI-based correlation analysis
Dangerousness index calculation for products and ingredients
Continuous updating as new data streams in

The ultimate goal:
Provide early warning detection.
Allow research prioritization.
Enable regulation or labeling of dangerous consumer products.
   Advantages over Prior Art or Attempts

Compared to traditional research this invention will enable at minimal extra cost:
1. Millions of hypothetical studies run automatically.
2. Trillions of permutations of combinations of products, food, ingredients and health conditions tested at minimal extra cost and instantly instead of over multiyear studies that are normally required.
3. This invention will also prioritize studies, preventing ones without statistical support and routing research monies to better ones.
4. Faster, cheaper, broader discovery saving years and discovering positive and negative health correlations that would normally never be discovered due to the huge cost, time, expert personnel and isolation of conditions of the variable of a study being impossible.
5. The ability to detect harmful combinations of products that individually seem safe. It being impossible to combine many products or ingredients in any given study for a single or multiple health conditions.
6. Early warning trend tracking across product categories, ingredients, demographics, geographies, and time.
7. This invention revolutionizes public health surveillance and research prioritization.

### Objective: Save Tens of Millions of Lives

Annual cancer deaths:
~8.9 million worldwide

Annual global healthcare costs for cancer treatment:
~$1.38 trillion

U.S. cancer stats:
~1.8 million new cases annually
~$140.8 billion in treatment costs annually

If even 10%-20% of disease burden can be attributed to preventable causes identified through this invention:
Millions of lives could be saved each decade.
Trillions of dollars in medical costs could be avoided.
Massive human suffering would be alleviated due to deaths, treatments and the financial burdens of these.

Electronic Medical Record (EMR) adoption since 2014 now provides a strong foundation to build this system using real-world data.

### BRIEF DESCRIPTION OF THE DRAWINGS

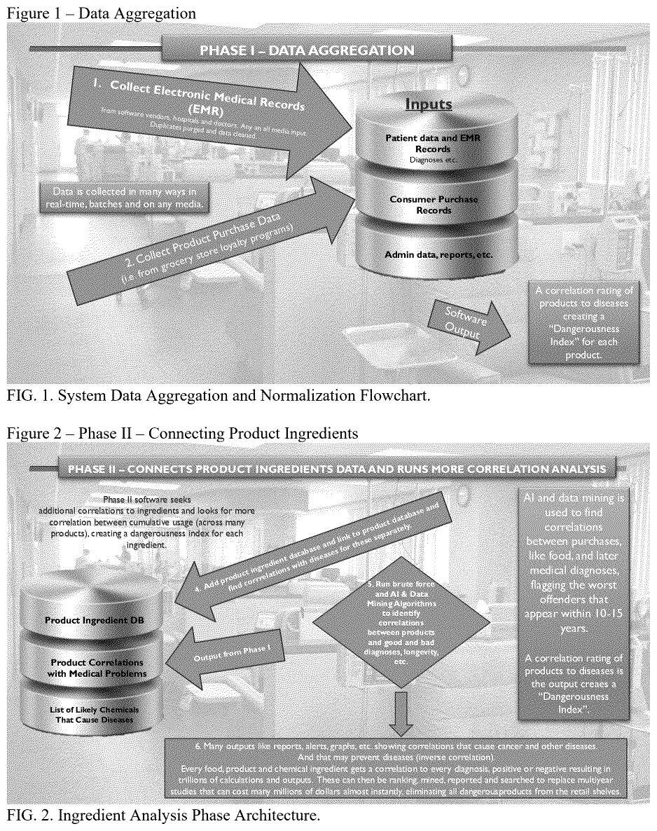

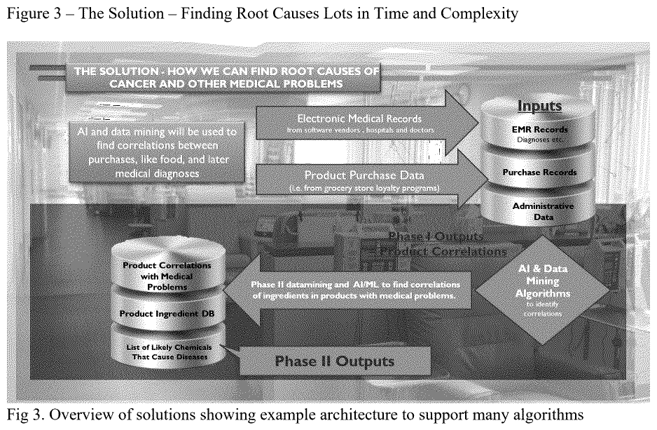

The system and process is illustrated through a phased approach:
Phase I shown in FIG. 1: Brute-force correlation of consumer product purchases with medical diagnoses using AI and statistical algorithms.
Phase II shown in FIG. 2: Ingredient-level analysis of products identified in Phase I to pinpoint problematic ingredients.
Phase III Shown in FIG. 3: Trend tracking, dangerousness index assignment, consumer alerting, and regulatory reporting.

Massive parallel processors and petabyte-scale storage systems are required to perform many trillions of comparisons, and to handle computer queries that can replace years of effort and millions of dollars to study today.

### DETAILED DESCRIPTION OF THE INVENTION

In addition to analyzing food and grocery-related records, the system described herein is further configured to assemble, normalize, and analyze Electronic Medical Records (EMRs) for the purpose of detecting statistical associations between various medical interventions and long-term health outcomes. These medical interventions may include pharmaceutical treatments, surgical procedures, alternative therapies, and other traditional or non-traditional treatment methods, whether approved or unapproved by regulatory agencies.

The system supports the analysis of multiple interventions per patient, recognizing that real-world treatment paths often involve combinations or sequences of different medical actions over time. For example, a patient may receive a pharmaceutical treatment followed by surgery and complementary therapies such as acupuncture or meditation, and the system is configured to correlate such longitudinal intervention patterns with later health outcomes.

Segmentation tools within the system allow cohort analysis based on any collected database field, including demographic, clinical, behavioral, or genetic attributes. Statistical association results may be quantified into correlation scores, dangerousness indices, or other health risk indicators, and may be published, reported, or transmitted through alert systems to consumers, businesses, or regulatory bodies.

FIGS. 1 through 3 illustrate the data ingestion, normalization, and analytical flow processes that support these expanded scientific study capabilities.

### Process Steps Details:

1. Collection of Medical Data Records—FIG. 1
Aggregate medical records from hospitals, clinics, insurers, and SaaS providers.
Standardize EMR records into a unified internal format.
De-duplicate records across providers.

2. Collection of Product Purchase Data—FIG. 1
Aggregate grocery store and retailer purchase records.
Capture product identifiers (UPC codes) linked to consumers via loyalty programs or credit card records.
Focus on products that are ingested, inhaled, or applied to skin.

3. Collection of Ingredient Data—FIG. 2
Map product UPC codes to ingredients.
Aggregate manufacturer ingredient disclosures.

4. Correlation Analysis FIGS. 2 and 3 (many variations) Conduct brute-force statistical comparisons across datasets. [0218] Apply Machine Learning (ML), Deep Learning (DL), or other AI techniques. [0219] (this line intentionally left blank)

5. Dangerousness Index Calculation FIG. 1 for product. FIG. 2 for ingredients.
Assign scores based on correlation strength.
Prioritize products and ingredients for deeper research and regulatory action.

6. Continuous Updating—A Function of Continuous Data Updating FIGS. 1, 2, and 3.
New purchase records and medical outcomes continuously added.
Dangerousness indices recalculated dynamically over time.
Alerts triggered for products or ingredients showing rising dangerousness trends.

7. Subgroup Analysis
Demographic filters: Age, gender, ethnicity, geography.
Genetic susceptibility analysis (optional, when genetic/DNA data available).

### Materials and Methods Employed in the Process

The system requires:
Massive cloud computing clusters (e.g., AWS, Azure) capable of trillions of calculations.
Petabyte-scale storage for purchase records, medical records, ingredient mappings.

Custom software for:
Parallelized brute-force correlation searching
Real-time dangerousness index updating
AI-assisted discovery of hidden product-disease relationships

Efficiency optimizations:
Early cutoff of low-correlation comparisons.
Dynamic loading and partitioning of datasets for high-speed processing.

### Example Outputs

Correlation scores linking products and ingredients to diseases.
Trend tracking over time.
Reports identifying the most dangerous and most beneficial products, ingredients, food categories, medical procedures, geographic locations, hospitals, etc.

### More Examples

Sample outputs from the system table:

| Product or | Potential | Deviation from Norm/ |
|---|---|---|
| Ingredient Consumed | Correlation Outcome | Probability (fake data) |
| High red meat | Increased arterial | +6.34%/p = 1.2% |
| consumption | sclerosis, angina, | |
| | early onset dementia | |
| Natural milk (1%) | Decreased osteoporosis | −7.4%/p = 0.2% |
| consumption | incidence | |
| High vegetable | Decreased heart disease | −4.0%/p = 2.2% |
| consumption | | |
| High berry | Lower cancer incidence | −6.34%/p = 1.2% |
| consumption | | |

(These are illustrative examples - actual results would emerge dynamically from the data.)

### Specific Potential Examples of the Process in Action

Examples of correlations the system might uncover:
Certain deodorants+certain soaps→Skin cancers
Air fresheners+cleaning sprays→Higher rates of asthma
Specific pesticide residues on imported produce→Higher rates of neurological disease
Excessive artificial sweetener consumption→Gut microbiome disruption→Higher diabetes risk
High vegetable intake reduces coronary diseases
Excessive fish or berry intake drives more cancer due to pesticides, mercury, etc.

The system enables the detection of correlations between combinations of products and diseases that would otherwise be invisible to human researchers. It may even contain exercise data when available to correlate positive and negative health outcomes with certain exercise.

1: (Core Aggregation): A system and method for aggregating and correlating Electronic Medical Records (EMRs) and consumer product purchase records, wherein said correlation allows identification of statistical associations between product usage patterns and subsequently diagnosed medical conditions. i.e. food, creams and lotions put on the skin, exercise, medical procedures, etc.
Dependent on claim 1:

1.1: The system of claim 1, wherein consumer product purchase records include loyalty program data, payment transaction data, or other personally identifiable purchase records from any source.

1.2: The system of claim 1, wherein the statistical correlation utilizes Machine Learning (ML), Deep Learning (DL), brute-force statistical methods, or Artificial Intelligence (AI) to detect positive or negative associations between health outcomes and the consumption or use of products, ingredients, or recorded activities, including cumulative effects arising from multiple exposures or repeated usage patterns, as reflected in EMR or other collected records.

1.3: The system of claim 1, wherein subgroup demographic or genetic filters are applied, including age, gender, ethnicity, occupation, and geographic location such that reports, real-time, alerts or other communications can be generated on correlations, as Dangerousness Index (DI) and positive and negative health effects can be examined by these filters, and are analyzed within said filtered subgroups.

1.4: The system of claim 1, wherein the system generates notice, reports and/or alerts to consumers based on identified high-risk product usage patterns, i.e. automatic emails, SMS or electronic communications.

1.5: The system of claim 1, wherein longitudinal tracking of purchase and health data is used to detect temporal trends in disease emergence using any filter or data field in any of the databases, i.e. zip code, ethnicity, product, medical procedure, activity.

1.6: The system of claim 1, wherein the Electronic Medical Records include hospital admission records, emergency room visit records, surgical intervention records, or other critical care events, and wherein said records are used in the correlation analysis to detect associations between consumer product usage and serious medical outcomes.

1.7: The system of claim 1, wherein a trend escalation module detects statistically significant increases in correlations between product usage and serious medical events, including hospital admissions and emergency room visits, and generates reports in any form or media, or automatically generates escalation alerts for regulatory bodies, healthcare providers, companies or consumers when predetermined risk thresholds are exceeded.

1.8: The system of claim 1, wherein alerts generated for consumers based on detected health risks are delivered via mobile application notifications, text messages (SMS), emails, wearable device alerts, or other electronic communication methods.

2: (Expanded Aggregation with Ingredients): A system and method for aggregating and correlating Electronic Medical Records (EMRs), consumer product purchase records, and product ingredient databases, wherein said correlation allows identification of statistical associations between specific ingredient exposures and subsequently diagnosed medical conditions.

2.1: The system of claim 2, wherein each consumer product's ingredients are mapped via a UPC code, or other unique identifying number, cross-referencing to a database of chemical components.

2.2: The system of claim 2, wherein Dangerousness Indices (“DI”) are calculated and continuously updated as new Electronic Medical Records and/or purchase records are added to the system, wherein each DI represents a scale showing the correlation strength to one or more negative or positive disease or health outcomes, and wherein a negative DI value indicates a positive health correlation and a positive DI value indicates a negative health correlation, or vice versa.

2.3: The system of claim 2, wherein subgroup analysis incorporates genetic susceptibility information based on DNA sequences, genomic markers, or other genetic profiling data, if available.

2.4: The system of claim 2, wherein products containing multiple correlated harmful (or helpful) ingredients are assigned cumulative dangerousness scores. i.e. a product/food contains multiple ingredients with high DI index and is ranked higher based on the total use of multiple products or ingredients, or even a potential volume of accumulated usage over time.

2.5: The system of claim 2, wherein dangerousness indices trigger automated reporting to regulatory agencies, insurers, researchers, healthcare providers or other corporate entities.

2.6: The system of claim 2, wherein consumer-facing applications display real-time product safety scores based on updated dangerousness indices.

2.7: The system of claim 2, wherein real-time computational models prioritize product-ingredient correlations by confidence interval, p-value, or Bayesian probability score.

3: (Alternative Treatments EMR Study): A system and method for assembling, normalizing, and analyzing Electronic Medical Records (EMRs), optionally supplemented with genetic or genomic data, over time to detect statistical associations between medical interventions, including pharmaceuticals, surgical procedures, alternative therapies, and/or non-traditional treatment methods, and the subsequent development or prevention of diseases or other health conditions, wherein the system enables population segmentation and cohort analysis based on any collected database field or demographic, clinical, or genetic characteristic.

3.1: The system of claim 3, wherein the medical interventions include one or more pharmaceutical treatments, surgical procedures, or other therapies or procedures, whether approved or unapproved by regulatory agencies.

3.2: The system of claim 3, wherein the medical interventions include alternative therapies such as yoga, acupuncture, meditation, herbal medicine, dietary changes, or other non-traditional methods not universally recognized by regulatory agencies.

3.3: The system of claim 3, wherein detected statistical associations are quantified into a correlation score, dangerousness index, or other risk indicator (with either positive or negative health outcomes), which is published, reported, or transmitted through alert systems to consumers, businesses, or regulatory bodies.
